(19) United States
(12) Patent Application Publication
(10) Pub. No.: US 2010/0318120 A1
(43) Pub. Date: Dec. 16, 2010

(54) HEMOSTATIC MATERIAL AND DELIVERY DEVICE

(76) Inventor: John Howard Gordon, Salt Lake City, UT (US)

Correspondence Address: CERAMATEC, INC., 2425 SOUTH 900 WEST, SALT LAKE CITY, UT, 84119, US

(21) Appl. No.: 12/815,589

(22) Filed: Jun. 15, 2010

Related U.S. Application Data
(60) Provisional application No. 61/187,232, filed on Jun. 15, 2009.
(60) Provisional application No. 61/327,209, filed on Apr. 23, 2010.

(51) Int. Cl. A61B 17/03; A61K 38/36

(52) U.S. Cl. 606/213; 514/13.6

(57) ABSTRACT

A hemostatic material, production method, delivery method, and apparatus are disclosed. The hemostatic material includes a peptide that preferentially selects exposed endothelial cells for bonding. The peptide is conjugated with a hemostatic agent (e.g., chitosan) to produce a peptide conjugated hemostatic agent. The peptide conjugated hemostatic agent is suspended in a flowable delivery medium that delivers the material to the endothelial cells to stop or reduce bleeding. An apparatus for delivering the hemostatic material includes a conformable covering for sealing off and maintaining an internal pressure in an injury cavity, a delivery port for delivering hemostatic material into the cavity, and a check valve that opens when a predetermined pressure is reached. Methods for producing the hemostatic material and using the apparatus are also disclosed herein.

### RELATED APPLICATIONS

This application claims priority to U.S. Provisional Patent No. 61/187,232 filed on Jun. 15, 2009 and entitled HEMOSTATIC MATERIAL AND DELIVERY METHOD and to U.S. Provisional Patent No. 61/327,209 filed on Apr. 23, 2010 and entitled DRESSING AND METHOD FOR DELIVERING HEMOSTATIC MATERIAL.

### BACKGROUND OF THE INVENTION

1. Field of the Invention

This invention relates to hemorrhage control. More particularly, this invention relates to novel hemostatic materials as well as devices for delivering the novel hemostatic materials to patients.

2. Background

The first action to be taken in treating a wound, where bleeding occurs, is to stop the bleeding. Stated in the abstract, the action of stopping the bleeding seems very straight forward. In practice, this is often not the case. The location of the wound, its size, accessibility to damaged vascular tissue, and a variety of additional issues, can make hemorrhage control extremely difficult. Although trained medical personnel and the right medical equipment can overcome some of these problems, wounds often occur at locations (e.g., on the battlefield, at remote locations, etc.) where the right medical personnel and medical equipment and supplies are not immediately available. Sometimes, medical personal and equipment, even if available, are still not sufficient.

Upon losing fifteen percent of its blood supply, the body begins to become adversely affected. At higher percentages of blood loss, such as around forty percent of its blood supply, death becomes a real concern. As a particularly forceful illustration of the scope of the problem, it is estimated that uncontrolled hemorrhaging is the leading cause of preventable combat deaths.

The application of pressure to seal off damaged vascular tissue is the most basic approach to stop bleeding. Simply pressing down on the wound or applying a tourniquet, however, will not be adequate in many situations. For many large and awkwardly-positioned wounds, these approaches are either too slow or entirely ineffectual. In some wounds, such as a wound to the abdomen, pressure points, where pressure can be applied to stop bleeding, are simply non-existent or insufficient. Additionally, stopping the blood flow in this way can damage or kill surrounding tissue—an unintended consequence for which the tourniquet is well known.

To address wounds that do not lend themselves to more basic, pressure-application approaches, hemostatic materials have been developed. These materials work to achieve hemostasis, the process by which the body stops bleeding. However, the different types of hemostatic materials currently available are not without their drawbacks and/or limitations.

A first type of hemostatic material, often classified as a ceramic hemostatic material, comprise inorganic, absorbent materials. Examples of these materials include QUIKCLOT® and WOUNDSTAT®. By absorbing liquid, these materials work to concentrate clotting agents and to increase the viscosity of blood flow. Although these materials have proven helpful to achieve hemostasis in large wounds, these inorganic materials are not readily absorbed by the body. Where a sufficiently large chunk of the material becomes dislodged, such as when the wound is cleaned, it may become lodged in the vascular system at various locations to cause thrombosis. This may restrict blood flow in the brain, the lungs, or other vital organs. Thrombosis arising from the use of these ceramic materials has led to death in some cases.

Another type of hemostatic material, known as a biopolymer, is bio-absorbable. The product HemCon®, whose primary active ingredient is the biopolymer chitosan, provides a good example of this type of material. Chitosan sticks indiscriminately to virtually anything that is wet within a wound. The chitosan works to achieve hemostasis by sticking to wet surfaces, such as damaged vascular tissue, and sealing off perforations, holes, and leaks. Although the chitosan in HemCon® is bio-absorbable, the non-specific way in which it sticks to wet surfaces in a wound is inefficient and can cause its own problems. Importantly, where blood egresses under sufficient pressure, the chitosan may be washed away and prevented from sticking to and sealing off damaged vascular tissue. The inability of chitosan to achieve hemostasis is common in dangerous arterial wounds.

In view of the foregoing, what are needed are hemostatic materials that are bio-absorbable and seek out damaged vascular tissues with specificity to achieve hemostasis in wounds regardless of the availability and use of pressure points. Production methods for these materials are also necessary. Further needed are apparatus and methods to deliver hemostatic materials to damaged vascular tissues without being washed away by the pressure of egressing blood flow and without damaging tissue or presenting a risk of thrombosis. Ideally, such materials, apparatus, and methods could be applied blindly on large and/or irregular wounds in the field by those with very little instruction or training.

### SUMMARY

The invention has been developed in response to the present state of the art and, in particular, in response to the problems and needs in the art that have not yet been fully solved by currently available structures and methods. Accordingly, the invention has been developed to provide structures and methods to overcome various shortcomings of the prior art. The features and advantages of the invention will become more fully apparent from the following description and appended claims, or may be learned by practice of the invention as set forth hereinafter.

In a first aspect of the invention, a flowable hemostatic material is disclosed. The flowable hemostatic material includes a peptide that preferentially selects exposed endothelial cells for bonding. The peptide is conjugated with a hemostatic agent, comprising at least one of a polymeric carbohydrate and a protein, which is suspended in a flowable delivery medium. Since endothelial cells are exposed at sites where bleeding occurs in an injury, the peptide conjugated hemostatic agent preferentially bonds to the site of bleeding so that the hemostatic agent can seal the wound. The flowable delivery medium allows the hemostatic agent to reach the site in question. Therefore, the amount of flowable delivery medium can be varied according to the embodiment so that the hemostatic agent can preferentially bond to the endothelial cells at a site where bleeding occurs in an injury cavity.

In some embodiments, the flowable hemostatic material also includes an absorbent, which is itself bio-absorbable, in an amount that supports the hemostatic agent in achieving hemostasis by reducing blood flow pressure. Blood flow pressure impedes the hemostatic agent from arriving at the site where bleeding occurs by flushing the hemostatic agent away. Additionally the absorbent supports hemostasis by concentrating natural agents in blood that foster clotting. In certain embodiments, fibrin may also be added to foster clotting.

In a second aspect of the invention, a method is disclosed for producing a flowable hemostatic material. The method involves conjugating a hemostatic agent with a peptide that preferentially selects exposed endothelial cells to produce a peptide conjugated hemostatic agent. The peptide conjugated hemostatic agent is suspended in a flowable delivery medium to make the selective hemostatic material flowable. In certain embodiments, the peptide conjugated hemostatic agent is formed by reacting the hemostatic agent with a linking molecule known as m-Maleimidobenzoyl-N-hydroxysulphosuccinide (MSB) and then reacting the MSB with a CGG amino-acid sequence in the peptide. In some embodiments, the method also involves adding the absorbent and/or the fibrin discussed above. In certain embodiments, amino acids that do not affect the ability of the peptide to preferentially bond to endothelial cells are removed.

In a third aspect of the invention, an apparatus for delivering a hemostatic material is disclosed. The apparatus includes a conformable covering for sealing off and maintaining an internal pressure in an injury cavity, a delivery port in the covering for delivering hemostatic material into the cavity, and a check valve in the covering that opens when a predetermined pressure is reached in the cavity. By sealing off the injury, the covering causes enough pressure to build in the injury cavity to stop or reduce the flow of blood so that the hemostatic material can be effective in achieving hemostasis. The check valve insures that the pressure does not reach dangerous levels or push unwanted materials into the wound. The predetermined pressure may approximate blood pressure.

In certain embodiments, the apparatus may also include a vent in the covering to release gases from the cavity, but prevent liquids and solids from escaping. In some embodiments, the apparatus may also include a release port to equalize pressure inside and outside of the cavity. The covering of the apparatus may also be embedded with fibers that act to prevent the covering from ballooning due to the internal pressure. The hemostatic material delivered by the apparatus may include the peptide conjugated hemostatic agent of the present invention or some other hemostatic material.

In a fourth aspect of the invention, a method is disclosed for delivering a flowable hemostatic material. The method involves applying hemostatic material through a delivery port in a conformable covering sealing off and maintaining an internal pressure in an injury cavity. The method also involves examining a check valve in the covering for egressing hemostatic material. When fluids egress from the check valve, the internal pressure is sufficient to stem the blood flow so that hemostatic material can achieve hemostasis. If fluids are not egressing from the check valve, the method continues by applying more hemostatic material.

In certain embodiments, the method involves allowing the cavity to experience the internal pressure for a predetermined period of time once fluids egress from the check valve. The method may also involve releasing the internal pressure through a pressure release port after the predetermined time and examining the release port in the covering for egressing fluids. The method may also involve repeating the preceding steps until fluids substantially cease to egress from the release port. Depending on the embodiment, the hemostatic material involved may include the peptide conjugated hemostatic agent, as described above.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1 is a high-level, schematic depiction, with expanded views, of a flowable hemostatic material in accordance with the invention;

FIG. 2 is a high-level, schematic depiction, of the hemostatic material working to achieve hemostasis through its constitutive elements bonding to exposed endothelial cells in damaged vascular tissue;

FIG. 3A is a high-level, schematic depiction of blood flow preventing a hemostatic material from achieving hemostasis;

FIG. 3B is a high-level, schematic depiction that is used to explain how the addition of an absorbent to the hemostatic material, in accordance with the present invention, can increase blood viscosity so that the hemostatic material can achieve hemostasis;

FIG. 4 is a high-level, schematic depiction, of a peptide that preferentially selects endothelial cells with which to bond and its constitutive amino acids, in accordance with the present invention;

FIG. 5A is a flow chart detailing one embodiment of a method for producing a hemostatic material in accordance with the present invention;

FIG. 5B is a flow chart detailing one embodiment of a method for producing a hemostatic material, with additional elements supporting the process of achieving hemostasis, in accordance with the present invention;

FIG. 6 is a high-level depiction of one embodiment of an apparatus for delivering a hemostatic material in accordance with the present invention;

FIG. 7 is a high-level depiction of one embodiment of a system for delivering a hemostatic material into an injury cavity to increase the internal pressure in that cavity so as to slow blood flow in order that the hemostatic material may achieve hemostasis;

FIG. 8 is a high-level depiction of one embodiment of a system for delivering a hemostatic material into an injury cavity to increase the internal pressure in that cavity so as to slow blood flow and to release the pressure before it can cause damage;

FIG. 9 is a flow chart detailing one embodiment of a method for delivering a hemostatic material to an injury cavity; and

FIG. 10 is a flow chart detailing one embodiment of a method for delivering a hemostatic material to an injury cavity and for verifying its effectiveness in achieving hemostasis.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

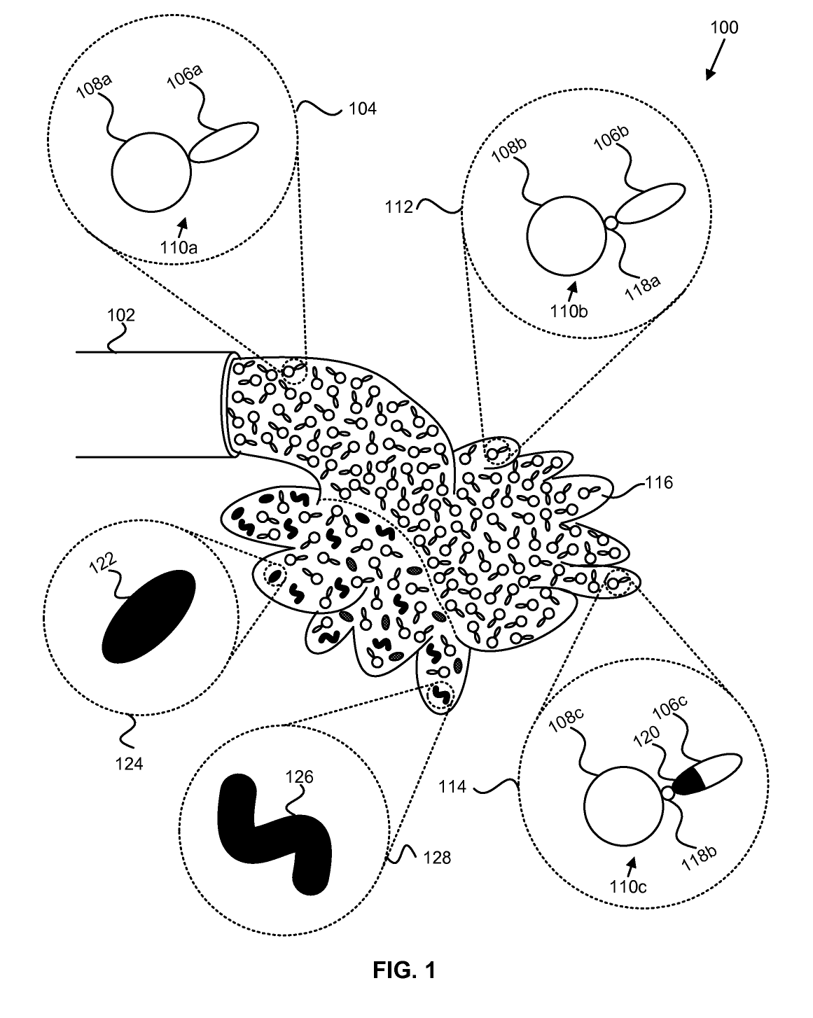

Referring to FIG. 1, one embodiment of a flowable hemostatic material 100 in accordance with the present invention is illustrated. FIG. 1 departs from the convention of depicting constitutive elements in proper proportions relative to one another so that the elements of the hemostatic material 100 may be depicted and so that the flowable nature of the hemostatic material 100 may be highlighted as an aspect of the present invention. In FIG. 1, the flowable hemostatic material 100 is depicted flowing from a conduit 102.

In the first expanded view 104, a peptide 106a is depicted that acts as a functional group to bind preferentially to a site where bleeding occurs, a site with exposed endothelial cells. The peptide 106a is conjugated with chitosan 108a to form a first peptide conjugated chitosan (PCC) 110a. The second expanded view 112 and the third expanded view 114 depict a second PCC 110b and a third PCC 110c, respectively. The first PCC 110a, second PCC 110b, and third PCC 110c are used to discuss alternative formations of PCC 110 consistent with the present invention.

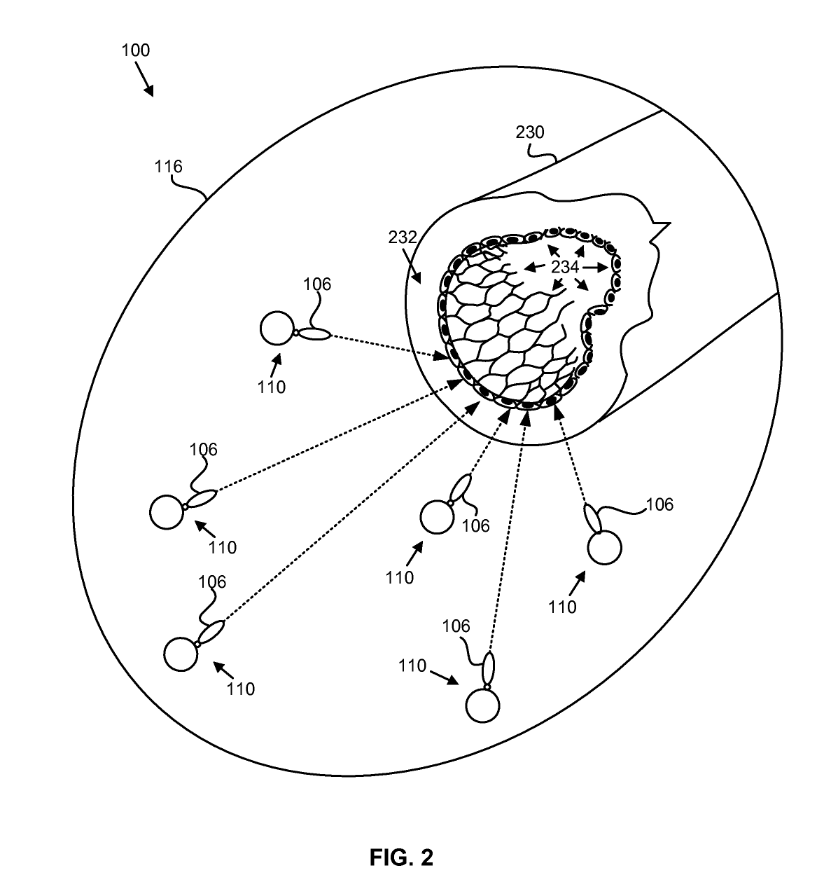

The peptides 106a, 106b, and 106c, (collectively 106) in the PCCs 110a, 110b, and 110c (collectively 110) preferentially selects exposed endothelial cells (see FIG. 2) to which to bond. Endothelial cells, which line the interior of blood vessels and arteries, are exposed at sites where bleeding occurs. Since endothelial cells have adhesion molecules that bond to laminin, the basal layer in blood vessels and arteries, various peptides naturally occurring in laminin can serve as the peptide 106 depicted in FIG. 1.

Certain peptides 106, naturally occurring in laminin, demonstrate a bonding affinity toward endothelial cells. Of these peptides 106, an additional subset demonstrates an aversion to bonding with cells other than endothelial cells. Studies have characterized the amino-acid-sequence structure of the laminin molecules, particularly laminin-1. Examples of sequences from laminin-1 consistent with the peptides 106 that can be used to practice the present invention may include C25, C38, C75, and C102 from the gamma region of laminin-1 and A25 and A99 from the alpha region. The group of peptides 106 comprising C25, C38, C75, and C102 provide examples of functional groups with both an affinity toward bonding with endothelial cells and an aversion to bonding with other kinds of cells.

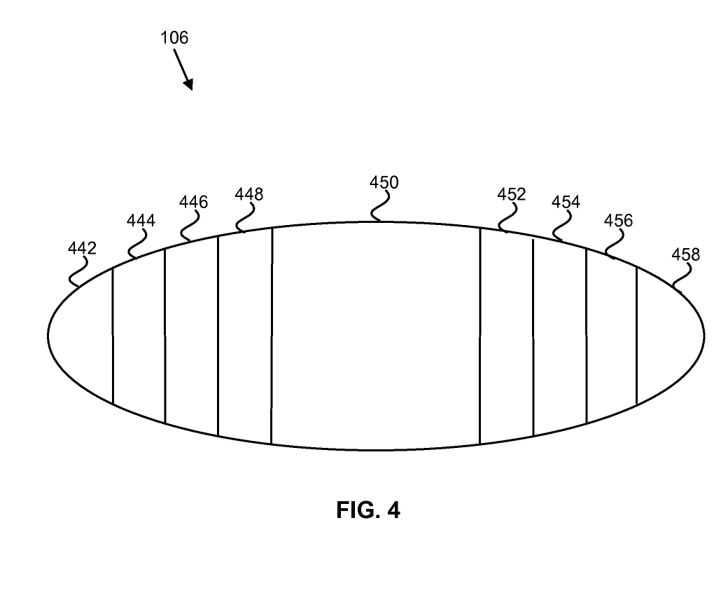

Additional examples of peptides 106 are also consistent with the present invention. For example, and without limitation, the following peptides 106 may also be employed: C16, C30, C64, A3, A10, A12, A13, A55, A65, A167, A203, and A208. In certain embodiments, the amino-acid sequence of the peptide 106 matches only a portion of the aforementioned peptide 106. In such embodiments, the residue of the peptide 106, may, among other possibilities, comprise a sequence of amino acids used to link the peptide 106 to the chitosan 108, as discussed in more detail below. Additional peptides 106 with alternative amino-acid sequences may be engineered in accordance with the present invention, as should become apparent to those of reasonable skill in the art through practice of the invention. One example of engineered sequences is discussed below with reference to FIG. 4.

In the second expanded view 112 of FIG. 1, the PCC 110b includes a linking molecule 118a. The linking molecule 118a facilitates the conjugating of the peptide 106b with the chitosan 108b. In some embodiments, the linking molecule 118a is first reacted with the chitosan 108b. In such embodiments, the linking molecule 118a is then reacted with the peptide 106b.

In the third expanded view 114, the peptide 106c includes a linking sequence 120 with the amino acid series CGG. The linking molecule 118b is reacted with the linking sequence 120 of the peptide 106c. The linking sequence 120 may be appended to any of the sequences for the previously mentioned peptides 106, or any other functional group 106 with an affinity for boding with endothelial cells. In certain embodiments, the linking molecules 118a and 118b (collectively 118) depicted in the second expanded view 112 and the third expanded view 114 respectively, is m-Maleimidobenzoyl-N-hydroxysulphosuccinimi (MBS).

The peptides 106 used to create the PCCs 110 for the hemostatic material 100 may be synthesized with Solid Phase Peptide Synthesis (SPPS). The ABI 433A peptide synthesizer provides an example of equipment capable of synthesizing such peptides. Additional procedures and equipment will be apparent to those of ordinary skill in the art.

Chitosan 108 is a very large, naturally occurring molecule that is bio-absorbable. As discussed in the background section, chitosan 108 can stick to an area where bleeding occurs, building up to seal off vascular tissue and achieve hemostasis. Chitosan 108, as conjugated with a peptide 106 to form PCC 110 in accordance with the present invention, may vary in molecular weight, depending on the embodiment.

Chitosan is one of many organic molecules that can be conjugated to a peptide. For example chitosan is from a group of polysaccharides which are polymeric carbohydrate structures including starch and glycogen, and structural polysaccharides such as cellulose and chitin. A peptide specific to binding to endothelial cells may also be conjugated with similar results to other structural organic molecules such as proteins, in particular keratin. Thus, in selected embodiments, hemostatic agents other than chitosan may be conjugated to a peptide in order to produce a peptide conjugated hemostatic agent in accordance with the invention. Thus, PCC represents just one type of peptide conjugated hemostatic agent and is not intended to be limiting.

PCCs 110, formed in accordance with the present invention, have the same attributes of their constitutive peptides 106. These PCCs 110 have an affinity for bonding with endothelial cells. In certain embodiments, these PCCs 110 also have an aversion to bonding with cells that are not endothelial cells. In other words, the PCCs 110 preferentially select exposed endothelial cells for bonding. The affinity of PCCs for endothelial cells, and in certain embodiments their aversion to bonding with cells that are not endothelial cells, is an important improvement over the prior art. Additional improvements, some of which are not directly highlighted, will become apparent in the body of the text below. As depicted, the PCCs 110 are suspended in a flowable delivery medium 116.

The PCCs 110 are suspended in a flowable delivery medium 116. Without limitation, the flowable delivery medium 116 may be water, an aqueous solution, a gel, alcohol, glycerin, or any other liquid, depending on the embodiment, capable of achieving flowability for the hemostatic material 100 at a desired viscosity, while satisfying additional concerns, such as concerns surrounding the bio-compatibility of the hemostatic material 100. The viscosity of the hemostatic material 100 may vary according to the embodiment. In certain embodiments, the viscosity is very low, allowing the hemostatic material 100, and the PCCs 110 therein, to access exposed endothelial cells, especially in irregular wounds. In certain embodiments, the viscosity is preferably higher so that the hemostatic material can be packed against exposed endothelial cells.

The flowability of the hemostatic material 100, as made possible by the flowable delivery medium 116, allows the PCCs 110 to more efficiently, and in certain embodiments selectively, deliver chitosan 108 to the sites where it is needed. Such sites, where bleeding occurs, are sites with exposed endothelial cells. (See FIG. 2). In certain embodiments, the flowable medium delivery 116 allows PCCs 110 to search out the exposed endothelial cells at the sites where they are needed within an injury cavity (not shown), even when applied “blindly,” or without topical specificity. All of these possibilities as they variously manifest themselves among different possible embodiments constitute improvements on the prior art.

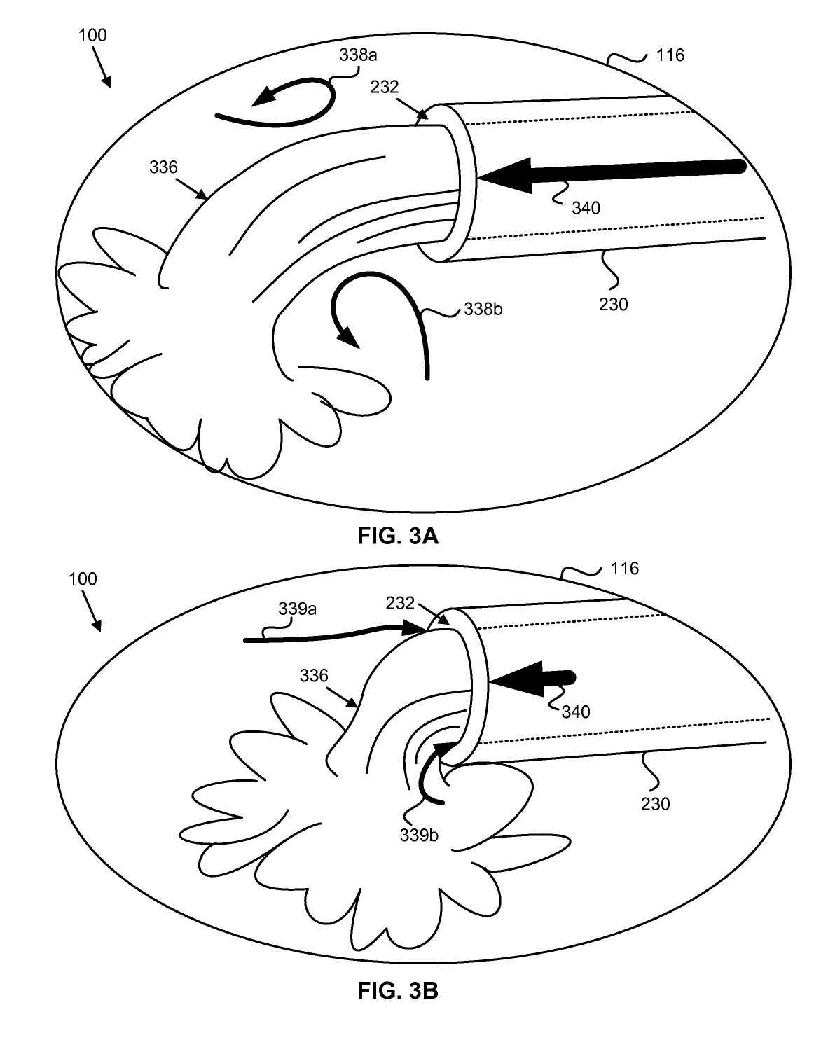

Although not necessary in all embodiments, in certain embodiments, the hemostatic material 100 also includes an absorbent 122, as depicted in the fourth expanded view 124. As with the PCCs 110, the absorbent 122 may be organic and bio-absorbable. The absorbent 122 may assist the PCCs 110 to achieve hemostasis by concentrating clotting agents in the blood as it removes water. Additionally, the absorbent 122 may assist in achieving hemostasis by increasing the viscosity of blood, allowing PCCs 110 to more easily reach exposed endothelial cells without being washed away by blood flow, as discussed with respect to FIG. 3A and FIG. 3B.

Unlike the ceramic absorbents discussed in the background section, the absorbent 122 of the present invention may be organic. Therefore, the absorbents 122, as do the PCCs 110, avoid the risk of leaving behind harmful particles that may lead to thrombosis or other problems. Types of absorbents 122 that may be employed include, without limitation, sodium polyacrylate (NaPa) and poly acrylamide (PAM), or a mixture thereof. NaPa has the advantage of being able to absorb 200 times its weight. Also, the resultant gel that forms when NaPa absorbs water can be dissolved by adding sodium chloride or saline when cleaning up the hemostatic material. PAM is unaffected by saline solutions and can remain in contact with tissues for a long time without leading to problems. Additional organic absorbents, known by those of ordinary skill in the art, may also be employed.

In certain embodiments, but not all embodiments, the hemostatic material 100 also may include fibrin 126, as depicted in the fifth expanded view 128. Fibrin 126 is a naturally occurring protein that fosters clotting in the blood. In certain embodiments, the hemostatic material 100 includes only PCCs 110; in other embodiments, the hemostatic material 100 includes PCCs 110 and absorbents 122; in some embodiments, the hemostatic material 100 includes PCCs 110, absorbents 122, and fibrin 126; and, in certain embodiments, the hemostatic material 100 includes PCCs 110 and fibrin 126.

Referring to FIG. 2, one embodiment of a hemostatic material 100 is depicted in a way that facilitates explanation of certain aspects of the manner in which the hemostatic material 100 works to achieve hemostasis. The hemostatic material 100 includes PCCs 110 suspended in a flowable delivery medium 116. As with FIG. 1, for purposes of explanation, the PCCs 110 are enlarged and are not necessarily representative of concentrations found in typical embodiments. Additionally, other elements that may or may not be included in different embodiments of the invention, such as the absorbents 122 and the fibrin 126 depicted in FIG. 1, are not illustrated.

The flowable delivery medium 116 envelopes a vascular conduit 230 that may be a vein, an artery, a collection of these, or some other form of vascular tissue that conducts blood flow. The vascular conduit 230 has an opening 232 created when the vascular conduit 230 is severed at the time the corresponding wound (not shown) was received. Lining the interior of the vascular conduit 230 are endothelial cells 234 that are exposed at the opening 232.

The PCCs 110 may be substantially similar to any of the PCCs 110a -110c discussed with reference to FIG. 1. The PCCs are depicted 110 with their constitutive peptides 106. The peptides 106 are oriented toward the endothelial cells 234 with arrows connecting various PCCs 110 to various endothelial cells 234 to indicate the preferential bonding between PCCs 110 and exposed endothelial cells 234. The flowable delivery medium 116 allows the PCCs 110 to become distributed in an injury cavity (not shown) so that they can come into contact with endothelial cells 234 where the PCCs 110 can act to reduce or seal off blood flow. Multiple PCCs 110 may bond to a single endothelial cell.

Referring to FIG. 3A, a hemostatic material 100 is illustrated in its interactions with a flow 336 of blood originating from a vascular conduit 230. The current 336 and the vascular conduit 230 are enveloped in the hemostatic material 100. In FIG. 3A, although flowable delivery medium 116 of the hemostatic material 100 is depicted, other constitutive elements are not illustrated for ease of explanation.

However, possible flow patterns 338a, 338b for PCC 110 are depicted. The current 336 of blood flow egresses under a pressure 340 proportional to the length of the arrow used to depict the pressure 340 for purposes of comparison with FIG. 3B. The pressure 340 of the current 336 of blood causes the flow patterns 338a, 338b to sweep the PCC away from the opening 232 in the vascular conduit 230, preventing the PCC from bonding with endothelial cells (not shown) found at the opening 232.

The presence of an absorbent 122 similar to the absorbent 122 discussed with respect to FIG. 1 leads to a different result in FIG. 3B. Referring to FIG. 3B, a hemostatic material 100 is illustrated in its interactions with a current 336 of blood flowing from a vascular conduit 230. Once again, of the elements of the hemostatic material 100, only the flowable delivery medium 116 is illustrated. However, the absorbent 122 component, which is not depicted, may play an important role in determining the pressure 340 causing the current 336 of blood to flow out of the opening 232 of the vascular conduit 230.

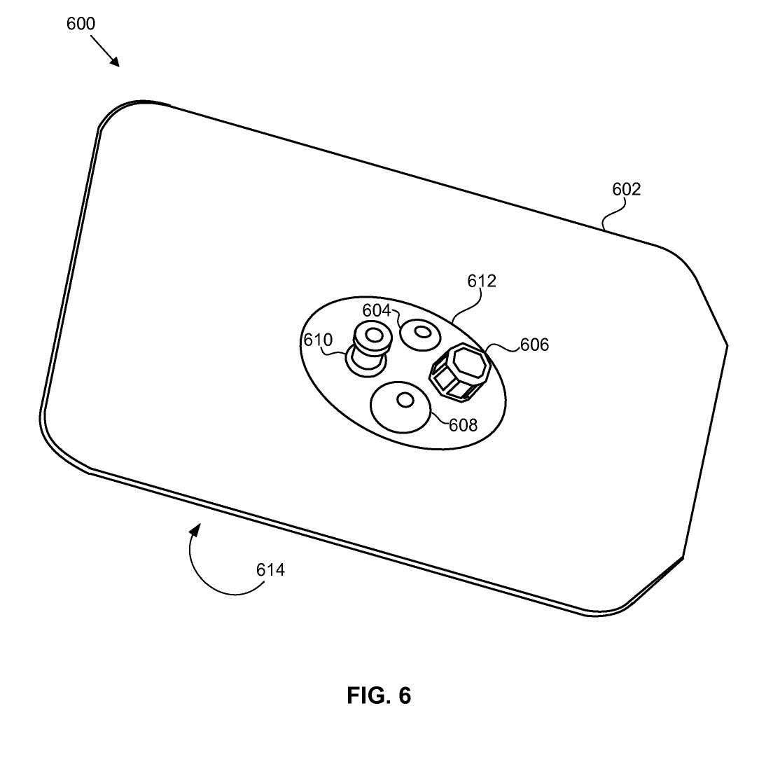

As indicated by the shortened length of the pressure 340 in FIG. 3B, the pressure 340 acting on the current 336 of blood depicted in FIG. 3B is much less than the pressure 340 acting on the current 336 of blood depicted in FIG. 3A. The reduced pressure 340 is the result of an increase in blood viscosity caused by absorbent (not shown) present in the hemostatic material 100. As the absorbent removes water from the blood, the viscosity increases. The reduced pressure 340 acting on the current 336 of blood as it egresses from the opening 232 in the vascular conduit 230 allows PCC (not shown) to diffuse to the opening 232, as indicated by the flow patterns 339a, 339b depicted in FIG. 3B. At the opening 232, the PCC is able to bond with endothelial cells (not shown) found in this area. FIG. 3B depicts one way in which the absorbent discussed with respect to FIG. 1 supports PCC in achieving hemostasis. However, in some situations, especially with arterial wounds, the absorbent 122 may not be sufficient to reduce the pressure 340 on the current 336 of blood. For such situations, additional measures consistent with the present invention may be employed, as explained in association with FIGS. 6 through 10.

Referring to FIG. 4, one embodiment of a peptide 106 in accordance with the present invention is illustrated. The peptide 106 includes a number of amino acids 442-458. A functional group 450 of amino acids is located in the center of the peptide 106. On either side, single amino acids 442-448, 452-458 are illustrated.

The functional group 450 comprises the amino acids responsible for preferentially selecting endothelial cells to which to bond. In certain embodiments, as explained with reference to the third expanded view 114 in FIG. 1, the first three amino acids 442-448 are a CGG series used to link the peptide 106 to a chitosan 108. However, in many embodiments, the single amino acids 442-448 and 452-458 are amino acids present in naturally occurring peptides 106 that preferentially select endothelial cells for bonding, as discussed with respect to FIG. 1.

Many of these single amino acids 442-448, 452-458 can be removed, or not added during the peptide synthesis process, without unduly influencing the ability of the peptide to preferentially select endothelial cells for bonding. In certain embodiments, a number of these single amino acids 442-448, 452-458 are removed, or not utilized during the creation or synthesis of the peptide. The number removed and the side, or sides, from which they are removed may vary greatly from embodiment to embodiment. Removal of amino acids 442-448, 452-458 is highly desirable because it greatly reduces the cost of synthesizing peptides 106 used to create the hemostatic material of the present invention. Embodiments that remove amino acids 442-448, 452-458 provide one example of engineered peptides 106. Additional examples of engineered peptides 106 are known to those of ordinary skill in the art.

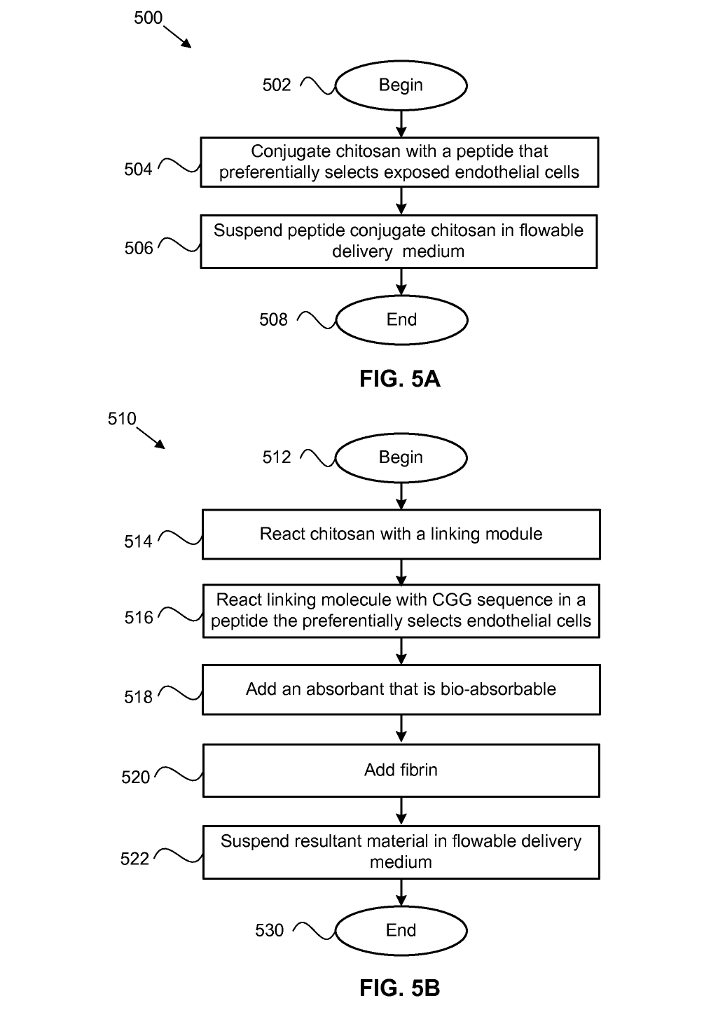

FIGS. 5A, 5B, 9, and 10 provide flow charts that set forth the logical structure of the methods of the present invention. The orders depicted in the flow charts are only indicative of particular embodiments of the present invention. The orders need not be observed in all embodiments of the invention and are included only for the purposes of illustrating the particular embodiments. The ordering is also ambiguous with respect to time in the sense that steps may occur concurrently or after a wait period. Additional steps may be added that are in keeping with the overall logical structure of the invention.

Referring to FIG. 5A, one embodiment of a method 500 for producing a hemostatic material in accordance with the present invention is illustrated. The method 500 begins 502 by conjugating 504 chitosan with a peptide that preferentially selects endothelial cells. Various types of peptide 106 and chitosan 108, as well as approaches for conjugating them, are discussed above in reference to FIG. 1. The method 500 continues by suspending 506 the peptide conjugated chitosan (PCC) in a flowable delivery medium. Various types of flowable delivery media 116 and considerations governing amounts thereof are discussed above with respect to FIG. 1. The method 500 then ends 508.

Referring to FIG. 5B, another embodiment of a method 510 for producing a hemostatic material in accordance with the present invention is illustrated. The method 510 begins 512 by reacting 514 chitosan with a linking molecule. The linking molecule depends on the embodiment, but as discussed above with respect to the second expanded view 112 and the third expanded view 114 in FIG. 1, the linking molecule 118 may be m-Maleimidobenzoyl-N-hydroxysulphosuccinimi (MBS) in certain embodiments.

The method 510 also involves, in some embodiments, reacting 516 the linking molecule with a CGG 120 sequence in a peptide that preferentially selects endothelial cells, as discussed with respect to the third expanded view 114 of FIG. 1. In some embodiments, the method 510 also includes adding 518 an absorbent that is bio-absorbable to the hemostatic material. The method 510 may also include adding 520 fibrin in certain embodiments. Various types of, uses for, and considerations pertaining to chitosan, peptide, absorbent, and fibrin are discussed above with reference to FIG. 1.

Disregarding the flowable delivery medium to be discussed below, the overall proportions relative to the remaining constituents of the hemostatic material may vary widely depending on the embodiment. The following are some relative proportions that are included only to enable certain embodiments and are in no way restrictive. PCC may vary from 1 to 100 percent by weight; absorbent may vary from 1 to 90 percent by weight; and fibrin may vary from 1 to 90 percent by weight. Additionally, the resultant material is suspended 522 in a flowable delivery medium in a manner substantially similar to the step 506 discussed above with respect to FIG. 5A. The method 510 then ends 530.

Referring to FIG. 6, one embodiment of an apparatus 600 for delivering the hemostatic material described herein (as well as other conventional hemostatic materials) is illustrated. As shown, the apparatus 600 includes a conformable covering 602 comprising an interface array 612. The interface array 612 may include a delivery port 604 and a check valve 606. In certain embodiments, the interface array 612 may also include a vent 608 and/or a pressure-release port 610.

The conformable covering 602 is conformable in the sense that it may be placed over different regions of the body (not shown) and conform to the contours of the body in that region. The conformable covering 602 may, in some embodiments, be made of a flexible polymer. In additional embodiments, the conformable covering 602 may be made of, without limitation, polyethylene, polycarbonate, PVC, acrylic, PTFE, PFA, silicone, nylon, or layers of these materials. Dimensions of the conformable covering 602 may vary widely to cover different sizes of wounds or may be cut to fit a particular size wound. Additionally, the flexibility of the conformable covering 602 may vary widely, in its various embodiments, to conform to the contour of different regions of the body where wounds may be sustained.

In selected embodiments, the conformable covering 602 includes adhesive material (not shown) on its underside 614. Several possibilities exist for the adhesive material and will be apparent to those of ordinary skill in the art. Depending on the embodiment, the adhesive material may surround just the corners of the underside 614, may cover the entire underside 614, the entire underside 614 except the region beneath the interface array 612, or cover any other of a wide array of possibilities so that the conformable covering 602 seals off the wound and is capable of maintaining an internal pressure inside the cavity of the wound.

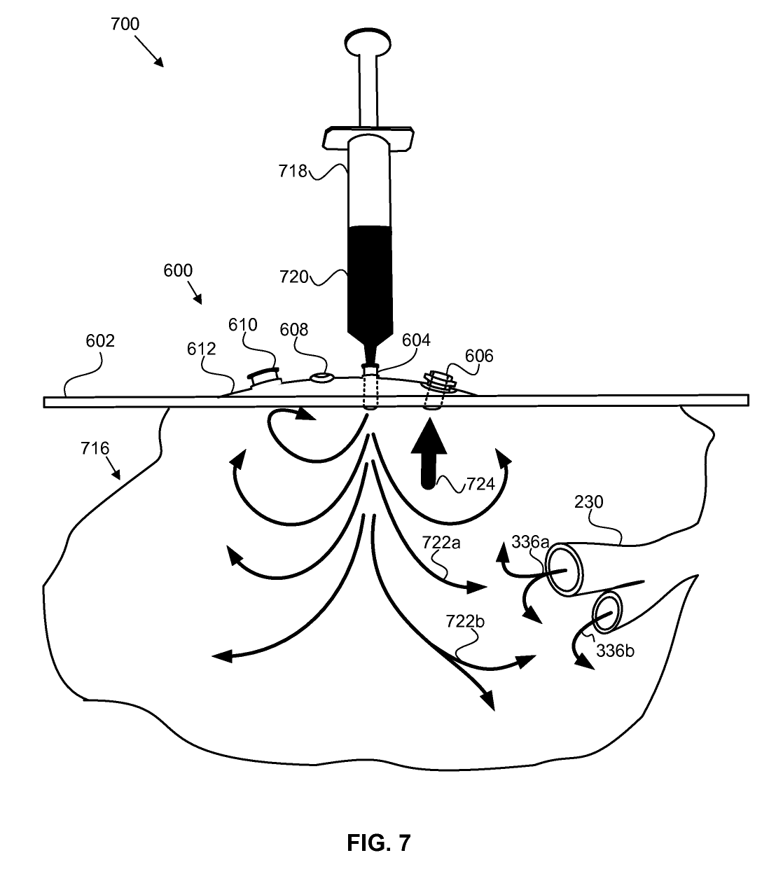

The magnitude of the internal pressure is discussed below with respect to FIG. 7. Although the conformable covering 602 is flexible in the sense that it can conform to the contours of the body, the conformable covering 602 may not be flexible in the sense that it is overly susceptible to stretching or “ballooning” in response to the internal pressure that it maintains. In some embodiments, fibers (not shown) are embedded within the conformable covering 602 to prevent such stretching. The fibers may be made of, for example, glass, metal, carbon, polyester, or other natural and/or synthetic materials.

The delivery port 604 disposed within the conformable covering 602 provides a conduit through which hemostatic material can be injected or delivered to an injury cavity (see FIG. 7) beneath the conformable covering. In some embodiments, the delivery port 604 may be as simple as conduit or passage. In certain embodiments, the delivery port 604 includes one of various types of Luer taper connections such as a Luer lock or Luer slip. In additional embodiments, any number of different fittings may be employed to foster one-way flow into the injury cavity.

In certain embodiments, the delivery port 604 is configured to receive a syringe (see FIG. 7) that can inject hemostatic material into the injury cavity. In some embodiments involving a syringe, the delivery port 604 prevents fluids from flowing out once the syringe is removed. However, the apparatus 600 is not limited to use with a syringe—any other system that can deliver hemostatic material into the injury cavity may also be used with the apparatus 600. The hemostatic material delivered through the delivery port 604 may be a hemostatic material consistent with the flowable hemostatic materials disclosed in association with FIGS. 1 through FIG. 5. However, the apparatus 600 may also be used with alternative hemostatic materials, such as those already existing in the prior art or modified versions of such materials, such as previously existing materials that are made sufficiently flowable for use with the apparatus 600.

The apparatus 600 also includes a check valve 606. The check valve 606 may have a predetermined cracking pressure. When the internal pressure maintained by the conformable covering 602 reaches the cracking pressure, the check valve 606 allows fluids, such as blood and hemostatic material, to egress out of the injury cavity. The relevance of this functionality, as provided by the check valve 606, is explained with reference to FIG. 7 in connection with values for the cracking pressure.

Some embodiments of an apparatus 600 include a vent 608. The vent 608 may allow gases to be released from the covering 602 but prevent liquids from escaping. For example, the vent 608 may include hydrophobic materials such as, without limitation, a polytetrafluoroethylene (PTFE) material like GOR-TEX® and/or a POREX® patch. Other materials apparent to those of ordinary skill in the art may also be used in the vent 608. In some embodiments, the vent 608 may be placed at a high point in the interface array 612 where gases accumulate.

In certain embodiments, the apparatus 600 includes a pressure-release port 610. When opened, the pressure-release port 610 equalizes the pressure in the injury cavity beneath the conformable covering 602 with the ambient pressure above the conformable covering 602. In certain embodiments, the pressure-release port 610 may allow not only gases, but fluids, to pass therethrough. Any number of valve systems may provide the functionalities of the pressure-release port 610 described herein.

Referring to FIG. 7, one embodiment of a system 700 for delivering hemostatic material 720 to an injury cavity 716 is illustrated. The system 700 includes an apparatus 600 comprising a conformable covering 602 and an interface array 612. The interface array 612 includes a delivery port 604, a check valve 606, a vent 608, and a pressure-release valve 610, as described in association with FIG. 6. The system 700 also includes a syringe 718 containing a hemostatic material 720. The hemostatic material 720 may be the hemostatic material 100 disclosed above with respect to FIG. 1 through FIG. 5, or some other hemostatic material.

In FIG. 7, a hemostatic material 720 is depicted being delivered into an injury cavity 716. Within the injury cavity 716, flow patterns 722a and 722b (collectively 722) are illustrated for the hemostatic material 720. Also, within the injury cavity 716, vascular conduit 230, which may include blood vessels and/or arteries, is illustrated with currents 336a and 336b (collectively 336) of blood flowing out of the vascular conduit 230.

As shown, the conformable covering 602 may be used to substantially seal off the injury cavity 716. As hemostatic material 720 is delivered into the injury cavity 716 and as the currents 336 of blood flowing from the vascular conduit 230 supply more blood to the injury cavity 716, the internal pressure 724, indicated by the upward pointing arrow 724, increases. The system 700 depicted in FIG. 7 serves to increase the internal pressure 724 to an appropriate level. At appropriate levels, the current 336 of blood is sufficiently slowed so that the hemostatic material 720 may come into contact with exposed endothelial cells (see FIG. 2) at the severed vascular conduit 230 without being flushed away by the currents 336.

Initially, the situation is similar to the situation depicted in FIG. 3A in which currents 336 egress from the vascular conduit 230 under sufficient pressure to flush away the hemostatic material 720, or as is the case in FIG. 3A, the PCCs 110 within the hemostatic material 100, which seal off the severed vascular conduit 230. As depicted in FIG. 7, the currents 336 egressing from the vascular conduit 230 are somewhat altered by the flow paths 722 of the hemostatic material 720, but still present an obstacle for the hemostatic material 720 to interacting with the endothelial cells (not shown) within the vascular conduit 230.

Although inclusion of an absorbent 122, as explained in association with FIGS. 1, 3A, and 3B, can sufficiently slow the current 336 of blood in certain wounds, this is not always sufficient. In some wounds, particularly wounds involving an artery, a more effective approach for slowing blood flow is required. The system 700 in FIG. 7 provides this approach. Since the conformable covering 602 seals off the internal cavity, the internal pressure 724 can be increased until it approaches and/or equals the pressure of the currents 336 of blood egressing from the vascular conduit 230. At these internal pressures 724, the currents 336 may slow and/or stop.

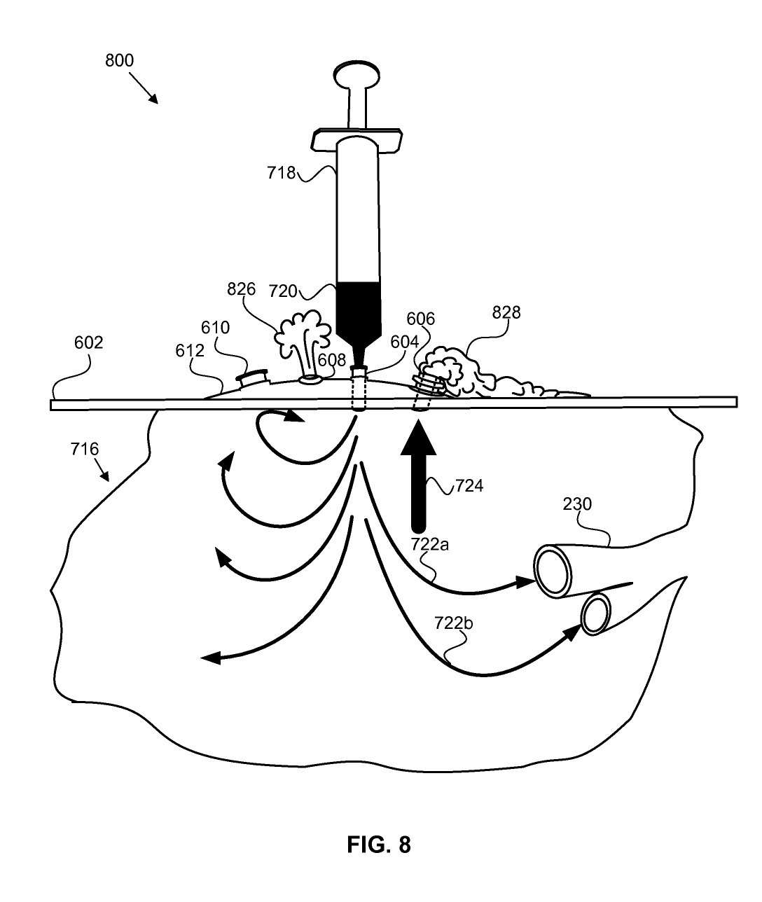

Regardless of the size of the artery, or the size or nature of the wound, the current 336 can be stopped by increasing the internal pressure 724 under the conformable covering 602 to the point that the internal pressure 724 equals the blood pressure. However, if the internal pressure 724 becomes too great, tissues surrounding the internal cavity 716 can become damaged from lack of blood flow. Furthermore, under internal pressures 724 that are too high, hemostatic material 720 or other materials (e.g., dirt, debris, etc.) can cause damage by entering damaged vascular tissue 230. The check valve 606 as previously discussed prevents the internal pressures 724 from becoming too high. The function of the check valve 606 will be explained in more detail in association with FIG. 8.

Referring to FIG. 8, as shown, more of the hemostatic material 720 has been injected into the injury cavity 716. Since more hemostatic material 720 has been delivered into the injury cavity 716, the internal pressure 724 has increased, as indicated by the increased length of the arrow used to depict the internal pressure 724. As the internal pressure increases, gasses 826, but not liquids or solids, are allowed to escape from the vent 608. Once the internal pressure has been increased sufficiently to substantially stop the flow of blood from the vascular conduit 230, the flow paths 722 are able to diffuse hemostatic material 720 to the vascular conduit 230 where the hemostatic material 720 can interact with the vascular conduit 230 to achieve hemostasis. In embodiments where the hemostatic material 100 contains PCCs as discussed in association with FIG. 1 through FIG. 5, the PCCs 110 bond to endothelial cells in the vascular conduit 230.

Assume that the internal pressure 724 in FIG. 8 exceeds the cracking pressure in the check valve 606. Since the cracking pressure has been exceeded, the check valve 606 will allow fluids 828 to egress from the internal cavity 716. These fluids 828 may include blood and/or hemostatic material 720. To prevent tissue damage and to prevent materials from being pushed into damaged vascular tissue 230, as discussed above, the cracking pressure of the check valve 606 may be set to approximate normal blood pressure. In certain cases, it may be necessary to inject several syringes 718 of hemostatic material 720 to achieve the cracking pressure.

As mentioned above, the cracking pressure of the check valve 606 may be set to a pressure equivalent to, or approximately equivalent to, the blood pressure in the body. In one embodiment, the pressure value is about 80 mm of mercury (Hg). In other embodiments, the pressure ranges from about 60 mm Hg to about 120 mm Hg. Nevertheless, values higher and lower than these may be used to achieve the goals of the present invention.

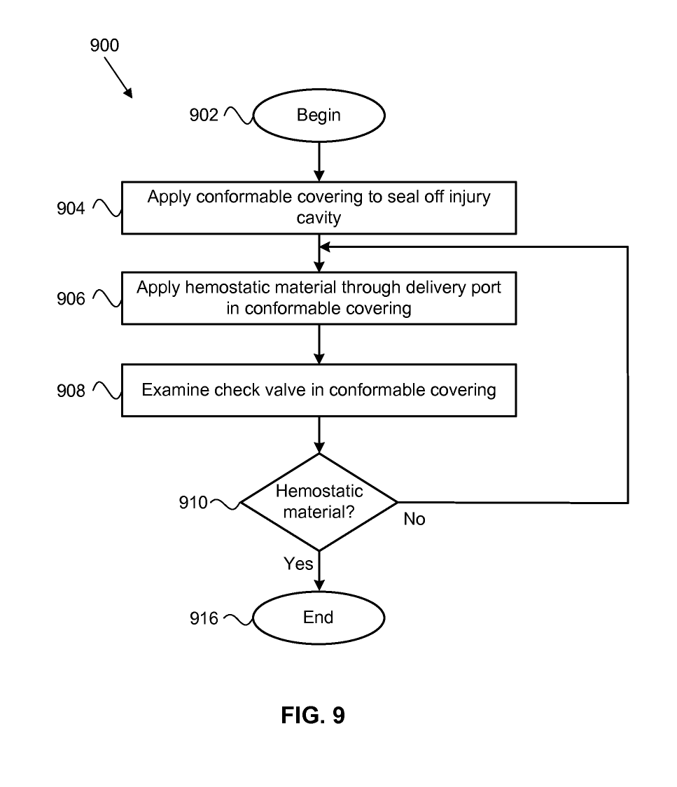

Referring to FIG. 9, one embodiment of a method 900 for delivering a hemostatic material to a patient is illustrated. The method 900 begins 902 by applying 904 a conformable covering 602 to a patient. The conformable covering 602 is applied to seal off an injury cavity so that an internal pressure can be maintained in the injury cavity. Once the injury cavity is sealed off, a hemostatic material is applied 906 through a delivery port 604 in the conformable covering 602. The hemostatic material may include the novel hemostatic material described in association with FIG. 1 through FIG. 5, or include some other hemostatic material the acts to achieve hemostasis. As the internal pressure beneath the conformable covering 602 increases, the flow of blood should reduce and allow the hemostatic material to achieve hemostasis without being washed away from the damaged vascular tissue.

As the hemostatic material is applied 906, a check valve 606 in the conformable covering is examined 908. A determination 910 is made as to whether hemostatic material is egressing from the check valve 606. When hemostatic material has egressed from the check valve 606, this signifies that the internal pressure has reached cracking pressure. This will ideally reduce the blood flow so that the hemostatic material can reach the damaged vascular tissue. If hemostatic material has not egressed from the check valve 606, the steps of applying 906 the hemostatic material, examining 908 the check valve 606, and determining 910 whether the hemostatic material is egressing from the check valve 606 may be repeated. Once the hemostatic material (or other fluids for that matter) have egressed from the check valve 606, then the method 900 ends 916.

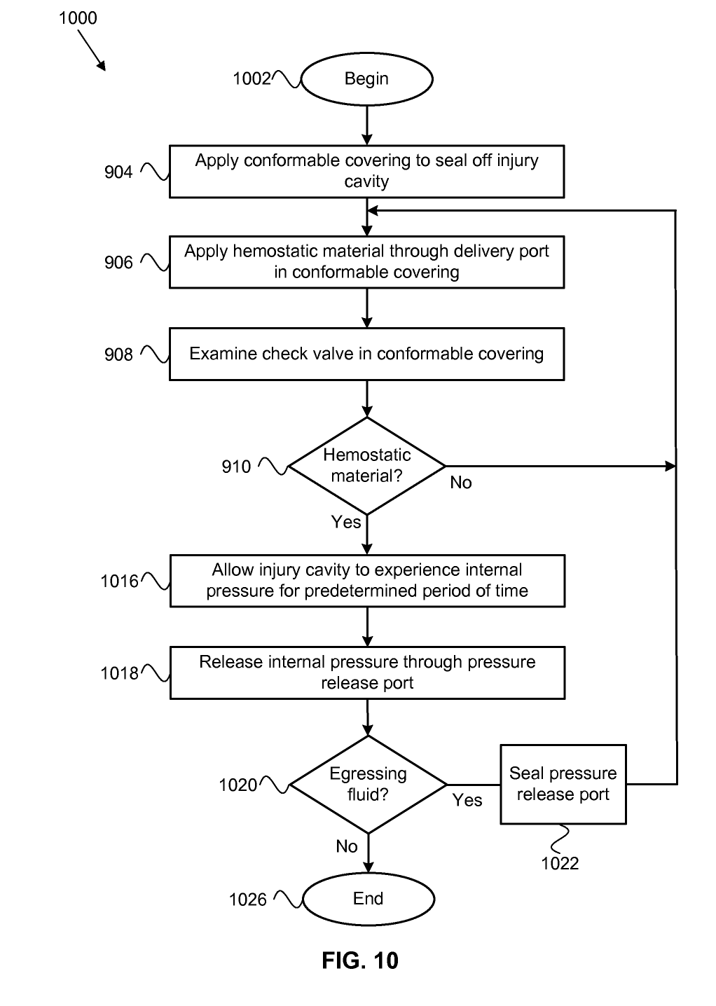

Referring to FIG. 10, one embodiment of a method 1000 for delivering a hemostatic material to a patient and verifying its effectiveness is illustrated. The method 1000 begins 1002 by applying 904 a conformable covering 602 to seal off an injury cavity, applying 906 hemostatic material through a delivery port in the conformable covering 602, and examining 908 a check valve 606 in the conformable covering 602. The method 1000 further includes determining 910 the presence of the hemostatic material egressing from the check valve 606. If the hemostatic material has egressed from the check valve 606, the method 1000 then allows 1016 the injury cavity to experience internal pressure for a predetermined period of time. The duration of the period of time may be calculated to allow the hemostatic material to achieve hemostasis. However, the duration of the period of time may vary widely depending on the hemostatic material and the injury to which it is applied.

After the predetermined period of time has passed, the internal pressure may be released 1018 through a pressure release port 610 in the conformable covering 602. The method 1000 then determines 1020 whether blood egresses from the pressure release port 610. If blood egresses from the pressure release port 610, then hemostasis has not been achieved. In such a case, the pressure release port is sealed 1022 and the process is repeated by returning to the application step 906. If substantially no blood egresses from the pressure release port 610, the method 1000 ends 1026.

The present invention may be embodied in other specific forms without departing from its basic principles or essential characteristics. The described embodiments are to be considered in all respects as illustrative and not restrictive. The scope of the invention is, therefore, indicated by the appended claims rather than by the foregoing description. All changes which come within the meaning and range of equivalency of the claims are to be embraced within their scope.

1. A hemostatic material in flowable form comprising:
a peptide that preferentially selects exposed endothelial cells for bonding;
a hemostatic agent, comprising at least one of a polymeric carbohydrate and a protein, conjugated with the peptide to produce a peptide conjugated hemostatic agent; and
a flowable delivery medium suspending the peptide conjugated hemostatic agent.

2. The hemostatic material of claim 1, wherein the flowable delivery medium is provided in an amount sufficient to make the hemostatic material sufficiently flowable so that the peptide conjugated hemostatic agent can preferentially bond to the endothelial cells at a site where bleeding occurs in an injury.

3. The hemostatic material of claim 1, further comprising an absorbent that is bio-absorbable.

4. The hemostatic material of claim 3, wherein the absorbent is selected from the group of organic polymer absorbents consisting of sodium polyacrylate, polyacrylamide, alkali polyacrylate, alkaline polyacrylate, polyacrylamine, and combinations thereof.

5. The hemostatic material of claim 1, further comprising fibrin.

6. The hemostatic material of claim 1, wherein the peptide comprises at least one of the following peptides: C16, C25, C30, C38, C64, C75, C102, A3, A10, A12, A13, A55, A65, A99, A167, A203, and A208.

7. The hemostatic material of claim 1, wherein:
the polymeric carbohydrate is selected from the group consisting of: polysaccharides, starch, glycogen, cellulose, chitin, and chitosan; and
the protein includes keratin.

8. A hemostatic material in flowable form comprising:
a peptide that preferentially selects exposed endothelial cells to which to bond at a site where bleeding occurs;
chitosan conjugated with the peptide to produce peptide conjugated chitosan (PCC) that acts to achieve hemostasis at the site; and
a flowable delivery medium suspending the PCC.

9. The hemostatic material of claim 8, wherein the flowable delivery medium is provided in an amount sufficient to make the hemostatic material sufficiently flowable so that the PCC can preferentially bond to the endothelial cells.

10. The hemostatic material of claim 8, further comprising a bio-absorbable absorbent in an amount that supports the PCC in achieving hemostasis.

11. The hemostatic material of claim 10, wherein the absorbent is selected from the group of organic polymer absorbents consisting of sodium polyacrylate, polyacrylamide, alkali polyacrylate, alkaline polyacrylate, polyacrylamine, and combinations thereof.

12. The hemostatic material of claim 8, further comprising bio-absorbable fibrin in an amount that supports the PCC in achieving hemostasis.

13. The hemostatic material of claim 8, wherein the peptide comprises at least one of the following peptides: C16, C25, C30, C38, C64, C75, C102, A3, A10, A12, A13, A55, A65, A99, A167, A203, and A208.

14. A method for producing a flowable hemostatic material, the method comprising:
conjugating a hemostatic agent, comprising at least one of a polymeric carbohydrate and a protein, with a peptide that preferentially selects exposed endothelial cells to produce a peptide conjugated hemostatic agent; and
suspending the peptide conjugated hemostatic agent in a flowable delivery medium to make the selective hemostatic material flowable.

15. The method of claim 14, wherein:
the polymeric carbohydrate is selected from the group consisting of: polysaccharides, starch, glycogen, cellulose, chitin, and chitosan; and
the protein includes keratin.

16. The method of claim 14, further comprising reacting the chitosan with a linking molecule known as m-Maleimidobenzoyl-N-hydroxysulphosuccinide (MSB).

17. The method of claim 16, further comprising reacting MSB reacted chitosan with a CGG amino-acid sequence in a peptide that preferentially selects endothelial cells for bonding.

18. The method of claim 14, further comprising adding an absorbent in an amount that supports the PCC in achieving hemostasis by reducing blood flow pressure.

19. The method of claim 14, further comprising adding bio-absorbable fibrin in an amount that supports the PCC in achieving hemostasis.

20. The method of claim 14, further comprising removing amino acids from the peptide without overly compromising the preferential selectivity of the peptide toward endothelial cells.

21. An apparatus for delivering a hemostatic material, the apparatus comprising:
a conformable covering for sealing off and maintaining an internal pressure in an injury cavity;
a delivery port in the covering for delivering a hemostatic material into the injury cavity; and
a check-valve in the covering that opens when a predetermined pressure is reached in the injury cavity.

22. The apparatus of claim 21, further comprising a vent in the covering to release gases from the injury cavity, wherein the vent prevents liquids and solids from escaping from the injury cavity.

23. The apparatus of claim 21, further comprising a pressure release port to equalize a pressure inside the injury cavity and a pressure outside the injury cavity when the pressure release port is opened.

24. The apparatus of claim 21, wherein the predetermined pressure is between about 60 mm of mercury and about 120 mm of mercury.

25. The apparatus of claim 21, wherein the predetermined pressure is about 80 mm of mercury.

26. The apparatus of claim 21, wherein the conformable covering is embedded with fibers that act to prevent the covering from stretching due to the internal pressure.

27. The apparatus of claim 21, wherein the hemostatic material comprises a peptide conjugated chitosan (PCC) suspended in a flowable delivery medium.

28. The apparatus of claim 27, wherein the hemostatic material further comprises an absorbent that is bio-absorbable.

29. The apparatus of claim 27, wherein the hemostatic material further comprises fibrin.

30. An apparatus for delivering a hemostatic material:
a conformable covering to maintain an internal pressure in an injury cavity to reduce blood flow and enable a flowable hemostatic material to make contact with an injury;
a delivery port in the covering for delivering the flowable hemostatic material into the injury cavity; and
a check-valve in the covering that opens when a predetermined pressure is reached in the injury cavity.

31. The apparatus of claim 30, wherein the predetermined pressure at which the check valve opens is selected to prevent damage to tissues in the injury cavity.

32. The apparatus of claim 30, further comprising a vent in the covering to release gases from the injury cavity while preventing liquids from escaping therefrom.

33. The apparatus of claim 30, further comprising a pressure release port to equalize a pressure inside the injury cavity and a pressure outside the injury cavity when the pressure release port is opened.

34. The apparatus of claim 30, wherein the hemostatic material comprises a hemostatic agent, comprising at least one of a polymeric carbohydrate and a protein, conjugated with the peptide and suspended in a flowable delivery medium.

35. The apparatus of claim 34, wherein the hemostatic material further comprises an absorbent that is selected from the group of organic polymer absorbents consisting of sodium polyacrylate, polyacrylamide, alkali polyacrylate, alkaline polyacrylate, polyacrylamine, and combinations thereof.

36. The apparatus of claim 34, wherein the hemostatic material further comprises fibrin in an amount that supports the hemostatic material in achieving hemostasis by fostering clotting.

37. The hemostatic material of claim 34, wherein the peptide comprises at least one of the following peptides: C16, C25, C30, C38, C64, C75, C102, A3, A10, A12, A13, A55, A65, A99, A167, A203, and A208.

38. A method for achieving hemostasis comprising:
applying hemostatic material through a delivery port in a conformable covering, the conformable covering sealing off and maintaining an internal pressure in an injury cavity;
examining a check valve in the covering for egressing hemostatic material; and
continuing to apply the hemostatic material until the hemostatic material egresses from the check valve.

39. The method of claim 38 further comprising:
allowing for the injury cavity to experience the internal pressure for a predetermined period of time;
releasing the internal pressure through a pressure release port;
examining the release port in the covering for egressing fluids;
repeating the preceding steps until fluids substantially cease to egress from the release port.

40. The method of claim 38, wherein the hemostatic material comprises peptide conjugated chitosan in a flowable form.
